(19) United States
(12) Patent Application Publication
(10) Pub. No.: US 2016/0113805 A1
(43) Pub. Date: Apr. 28, 2016

(54) ORTHOTIC AND ASSISTIVE DEVICES, SYSTEMS AND METHODS

(71) Applicant: Board of Regents of the University System of Georgia, by and on behalf of the University of Nor, Dahlonega, GA (US)

(72) Inventors: Terrie L. Millard, Dahlonega, GA (US); Jon Mehlferber, Dahlonega, GA (US); Alison Alhadeff, Dahlonega, GA (US); Enes Aganovic, Dahlonega, GA (US)

(21) Appl. No.: 14/923,254

(22) Filed: Oct. 26, 2015

Related U.S. Application Data
(60) Provisional application No. 62/068,505, filed on Oct. 24, 2014.

(51) Int. Cl. A61F 5/02; A61F 5/37

(52) U.S. Cl. CPC A61F 5/02; A61F 5/3784

(57) ABSTRACT

An orthotic and assistive device for persons with disabilities is provided. The device is configured to allow the user to safely and more fully participate in conventional activities, such as riding a horse and a bicycle. Components of the device support the user and maintain the user in a desired form. Components of the device are inexpensively and quickly produced by 3D printing. As such, components can be custom fit for each user to enhance the effectiveness of the device.

### CROSS-REFERENCE TO RELATED APPLICATIONS

This application claims the benefit of and priority to U.S. Provisional Patent 62/068505, filed Oct. 24, 2014, which is incorporated in its entirety in this document by reference.

### FIELD OF THE INVENTION

This invention relates generally to the field of orthotic and assistive devices for use in physical therapy by users with various neuromuscular dysfunction. More particularly, the present application relates to improved assistive, orthotic devices for use in hippotherapy, on a bicycle or other vehicle and on a flat surface like a floor or chair.

### BACKGROUND OF THE INVENTION

Lack of appropriate adaptations and equipment compromises the ability of people with special needs to participate in exercise and leisure activities alongside typically developing peers. Further, lack of appropriate adaptations and equipment can compromise the ability of people with special needs to safely and effectively engage in desired physical therapy sessions, such as hippotherapy. If a child is more actively involved in motor activities, motor learning and development may be enhanced. Manufacture of cost-effective and accessible adaptive equipment could enhance opportunities for physical therapy and for independent participation in normal physical and social activities.

Hippotherapy promotes gross motor skills for children with limited or atypical movement. The horse serves as a dynamic base of support encouraging patients to control and coordinate movement, improve symmetry of muscle activation and motor function. Side walkers are utilized to provide support and safety in keeping the patent on the horse. The support offered by a side walker has no external regulation and may be over-supportive or under-supportive. Moreover, in conventional hippotherapy, the side walker provides supports to keep the patient on the horse, which can be unsafe.

What is needed then are inexpensive, assistive, orthotic devices configured to enable users with special needs to safely participate in activities, such as bicycle riding, hippotherapy and the like.

### SUMMARY OF THE INVENTION

According to various embodiments, the present application is for an orthotic and assistive device for persons with disabilities. For example, a device is provided which enables a user to safely and more efficiently ride a horse such as during hippotherapy. In another example, a device is provided to inexpensively modify a conventional bicycle to allow a user to more safely and efficiently ride the bicycle. In one aspect, the orthotic and assistive device can be a low-cost orthotic and assistive device formed at least in part by 3D printing.

In one embodiment, the orthotic device can be configured for use in riding an animal such as a horse. According to one aspect, the device can comprise a torso support element to support the torso of the user therein, an animal engagement member to safely support the device on the animal, and a strut configured to couple the torso support element to the animal engagement member.

In use, the torso support element can be placed around the torso of a user so that the torso of the user extends through an opening of the torso support element. A tether or strap can be adjusted so that the opening has a predetermined size and thus, provides a desired level of support to the user. The distance between an upper edge of the torso support element and a portion of the animal engagement member can be adjusted to a predetermined height by sliding the torso support element relative to the strut. The user and the device can be placed on the animal, so that a bottom surface of the animal engagement member contacts the back of the animal. The user can then be led around by a side walker during a hippotherapy session.

In another embodiment, the orthotic device can be configured for use in riding a vehicle such as a bicycle. According to one aspect, the device can comprise a torso support element to support the torso of the user therein, a hand attachment member to couple the hand of the user to the handlebar of the bicycle and a foot attachment member to couple the foot of the user to the pedal of the bicycle. The device can further comprise a yoke configured to support the head and/or neck of the user in a desired position.

In use, the torso support element can be placed around the torso of a user so that the torso of the user extends through an opening of the torso support element. A tether and/or side members of the torso support element can be adjusted so that the opening has a predetermined size and thus, provides a desired level of support to the user. The hand of the user can be positioned in the hand attachment member and the hand attachment member can be coupled to the handlebar. Tethers, straps, and the like can be used to secure the hand and/or the hand attachment member in place. The foot of the user can be positioned in the foot attachment member and the foot attachment member can be coupled to the pedal. The distance between an upper edge of the foot attachment member and the pedal can be adjusted to a predetermined height by sliding a portion of the foot attachment member relative to the pedal. Tethers, straps, and the like can be used to secure the foot and/or the foot attachment member in place. The yoke can be positioned as desired around the head of the user and the user can pedal the bicycle as a conventional bicycle.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE DRAWINGS

The accompanying drawings, which are incorporated in and constitute a part of this specification, illustrate several embodiments of the invention and together with the description, serve to explain the principles of the invention.

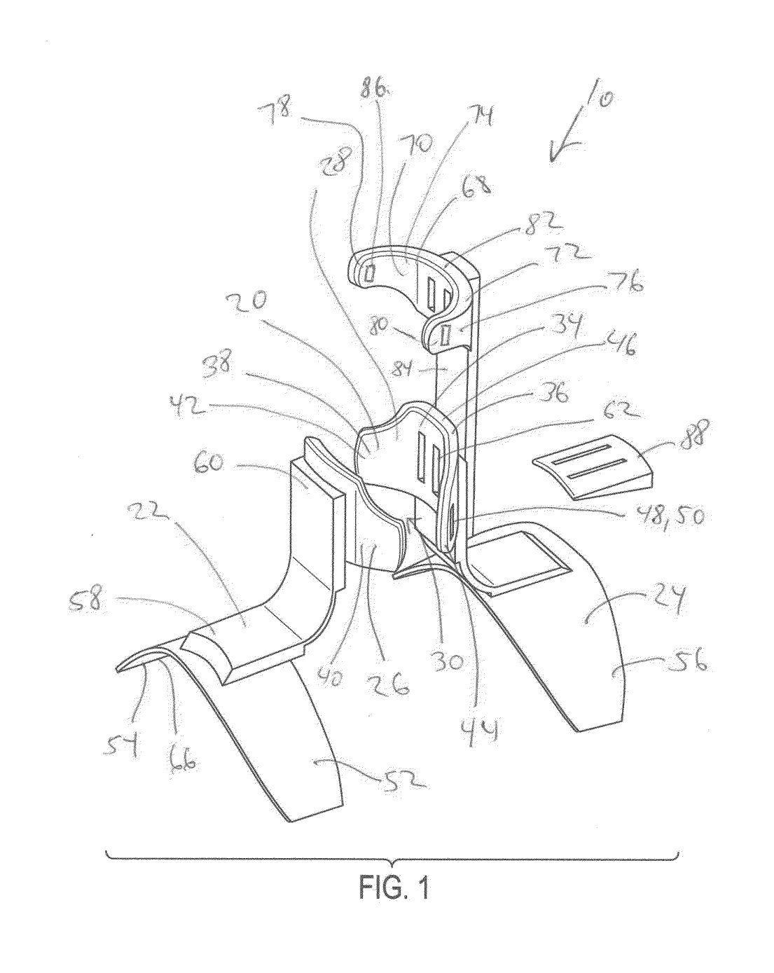

FIG. 1 is side perspective view of an embodiment of the orthotic and assistive device of the present application, showing a torso support element, at least one strut, and at least one animal engagement member, according to one aspect.

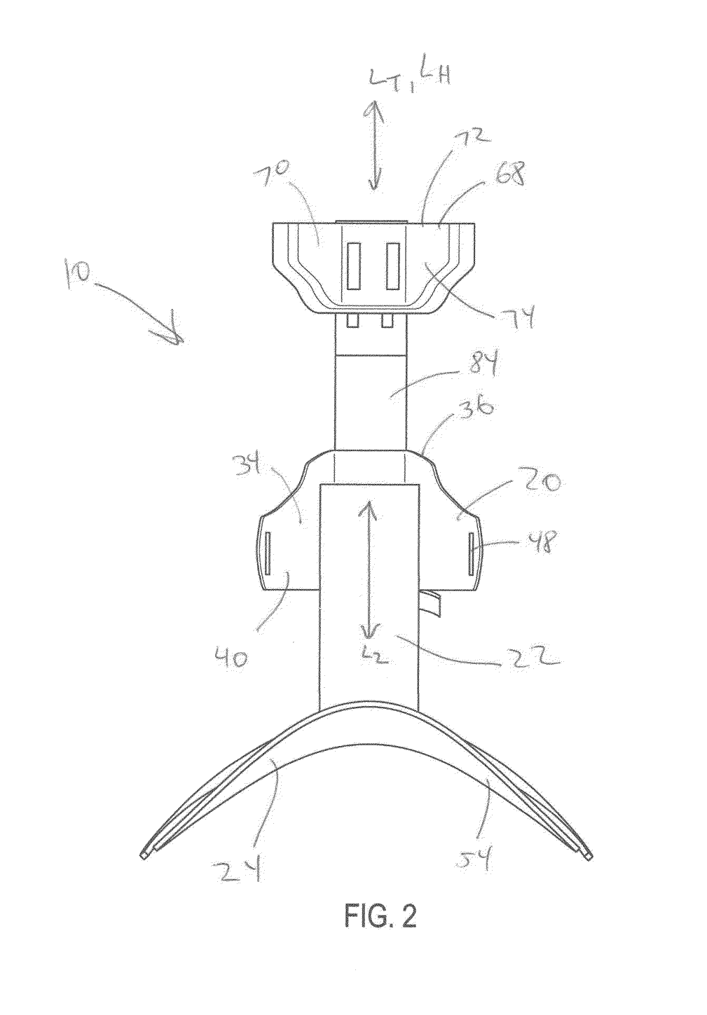

FIG. 2 is a front elevational view of the device of FIG. 1.

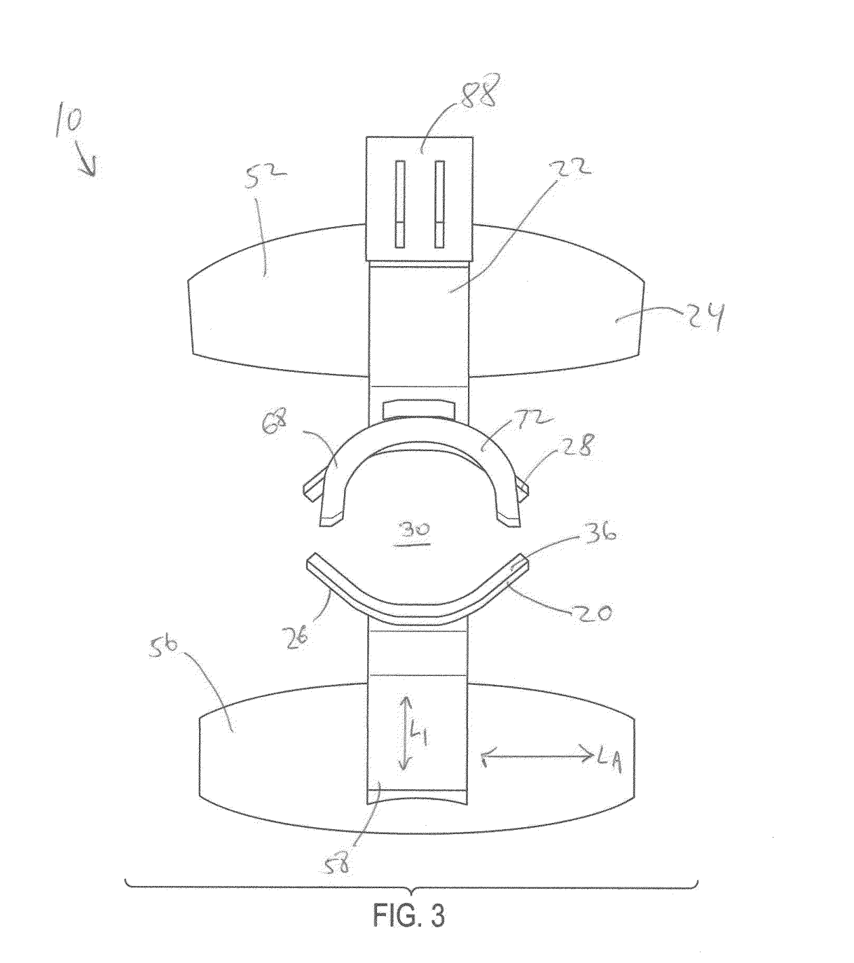

FIG. 3 is a top view of the device of FIG. 1

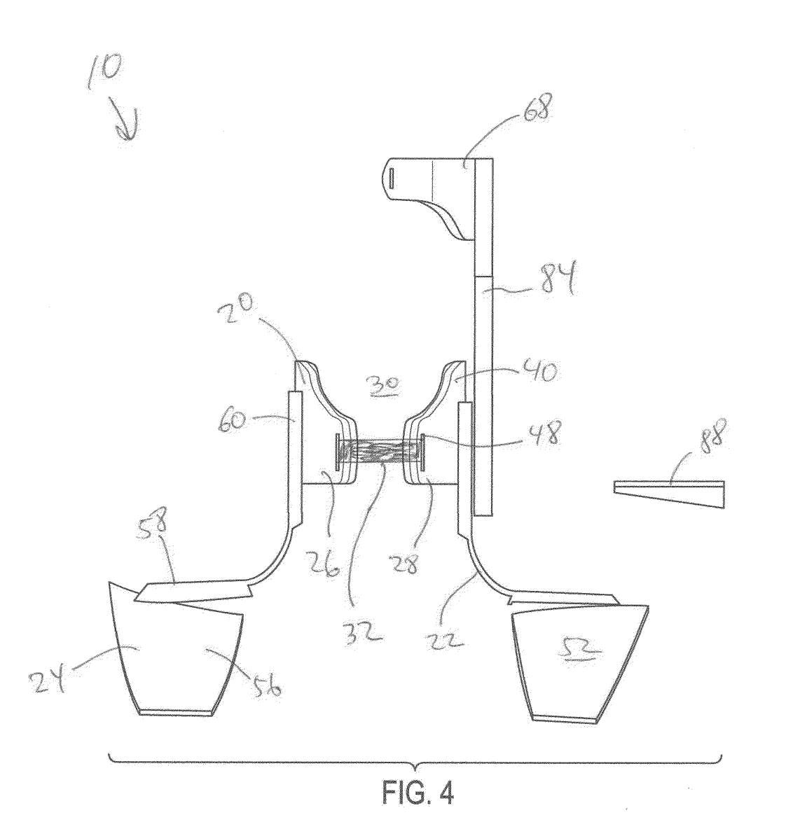

FIG. 4 is a side elevational view of the device of FIG. 1.

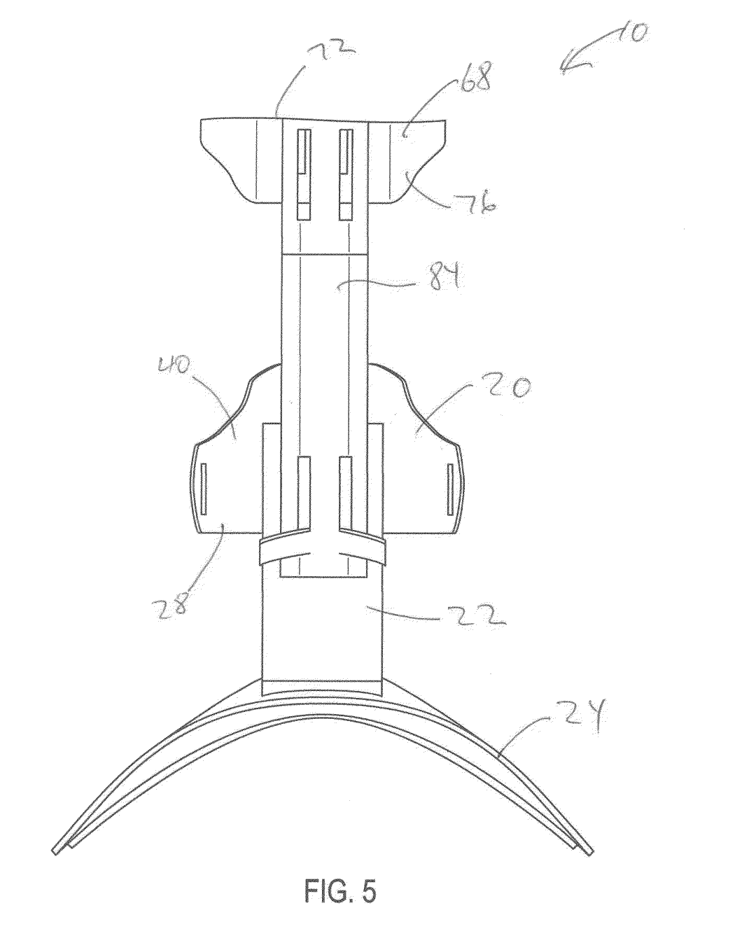

FIG. 5 is a rear elevational view of the device of FIG. 1.

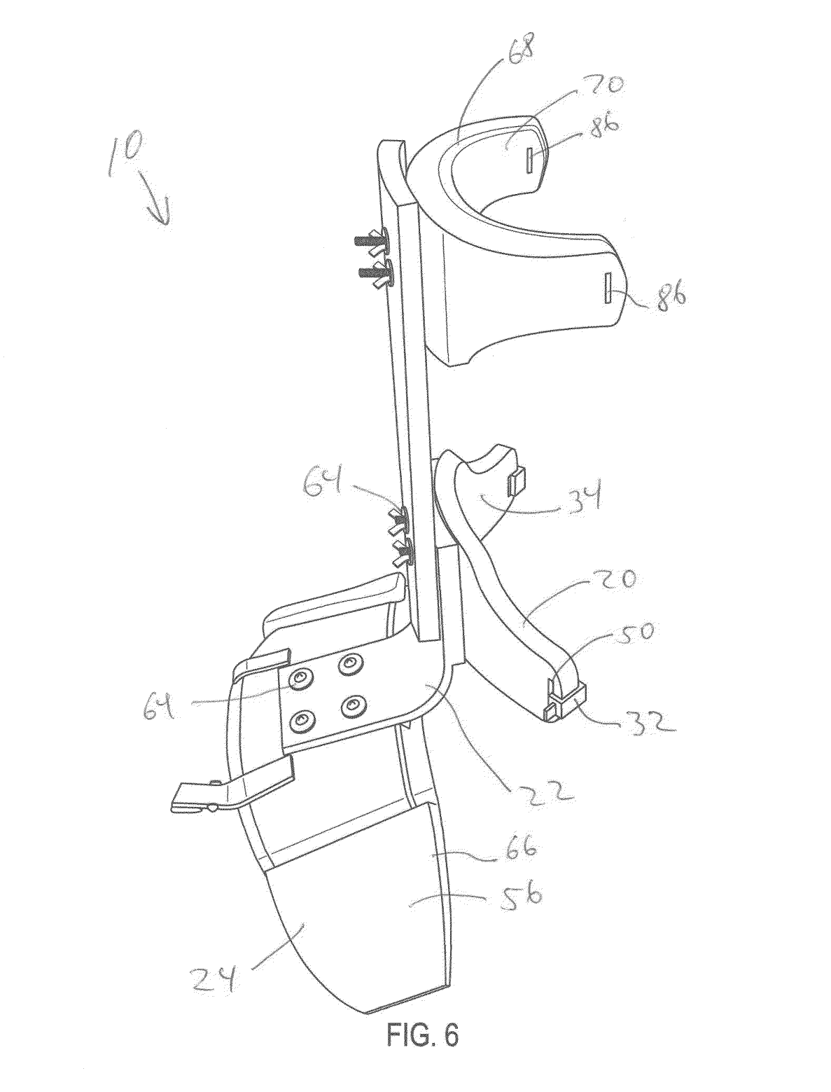

FIG. 6 is a perspective view of a portion of the device of FIG. 1.

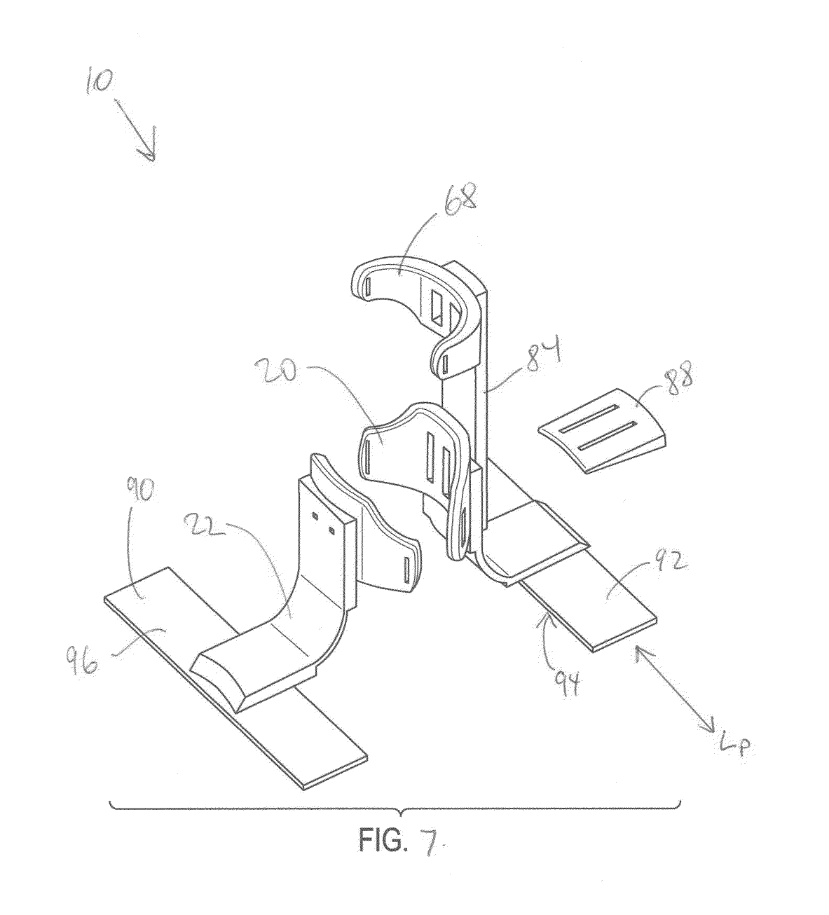

FIG. 7 is side perspective view of a second embodiment of the orthotic and assistive device of the present application, showing a torso support element, at least one strut, and at least one planar engagement member, according to one aspect.

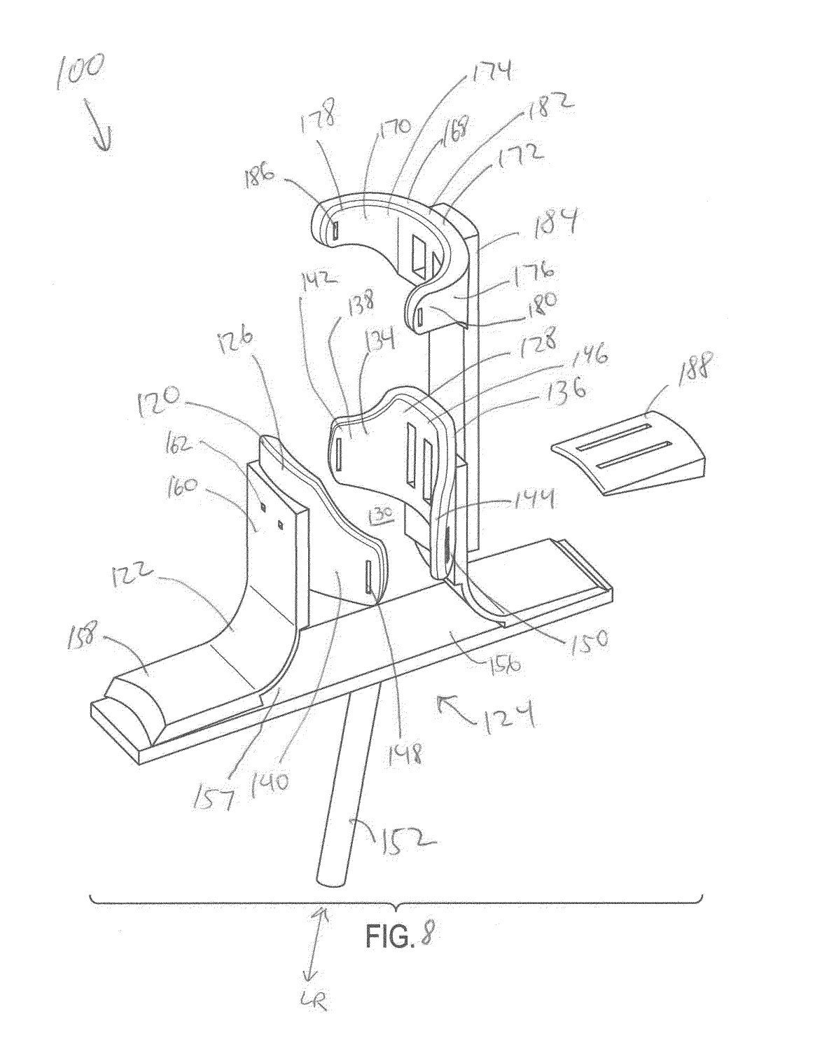

FIG. 8 is side perspective view of a third embodiment of the orthotic and assistive device of the present application, showing a torso support element, at least one strut, and at least one bicycle engagement member, according to one aspect.

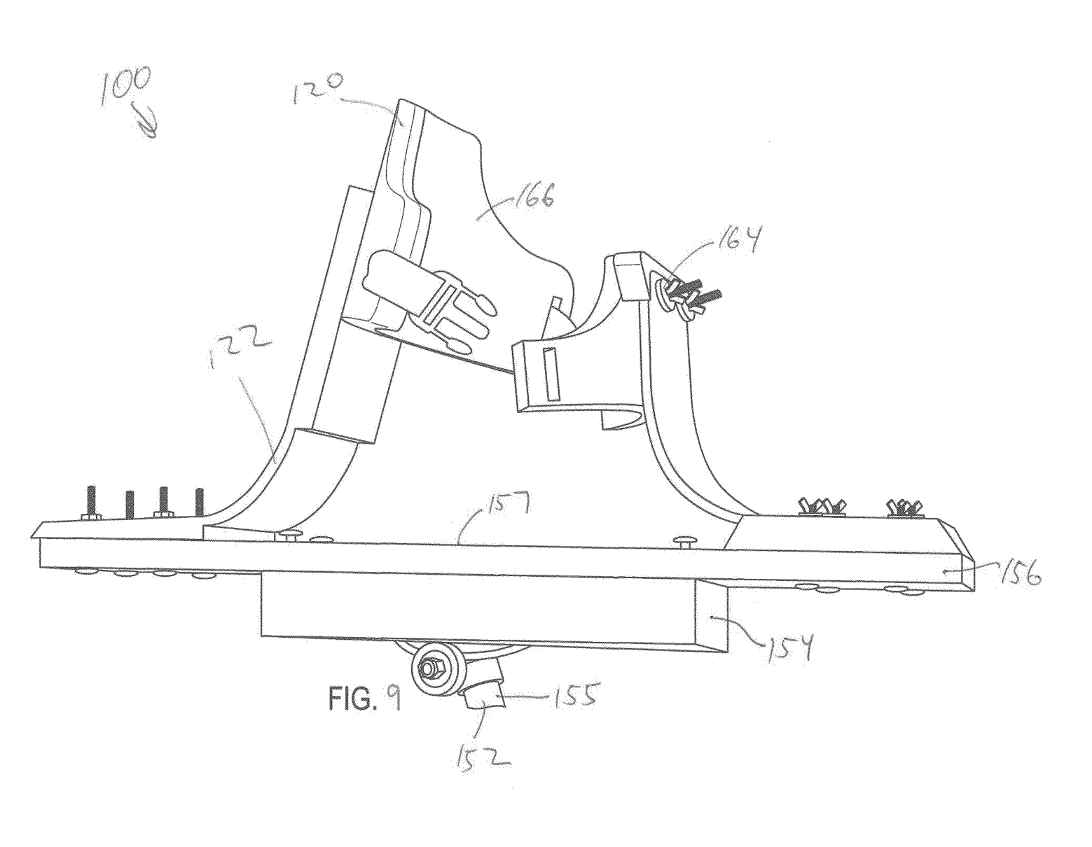

FIG. 9 is a side elevational view of the device of FIG. 8.

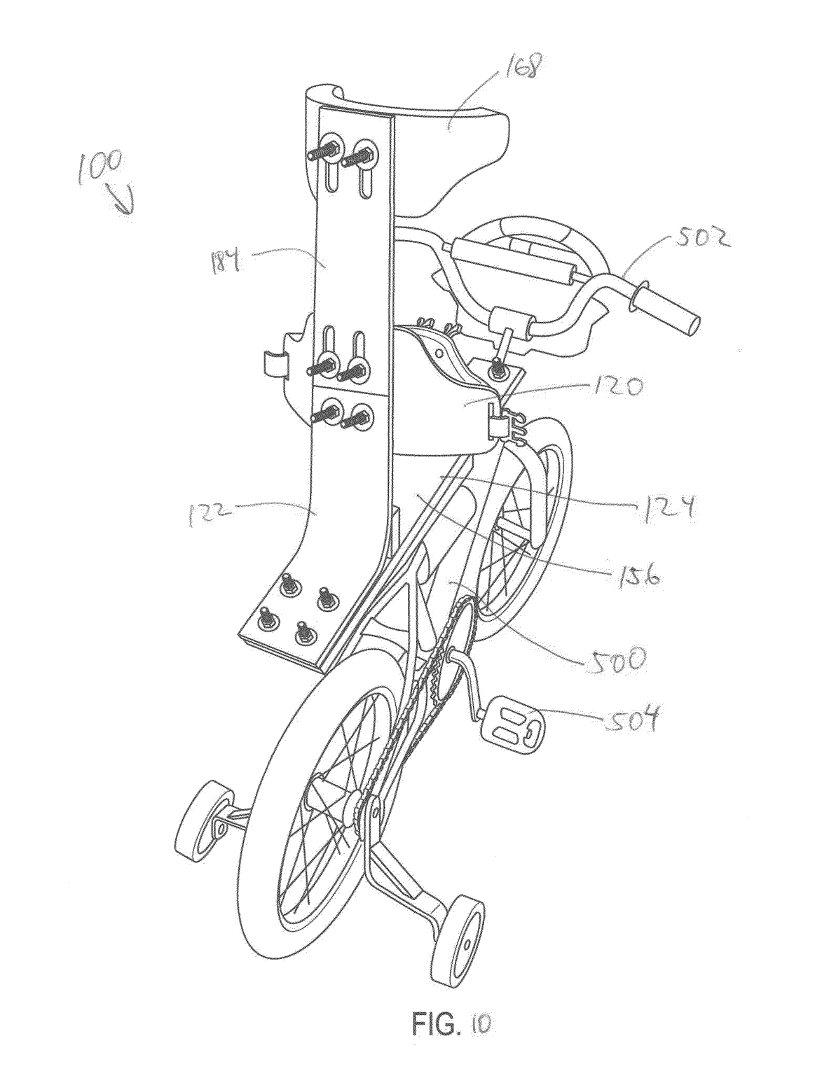

FIG. 10 is a rear perspective view of the orthotic and assistive device of FIG. 8 attached to a bicycle, according to one aspect.

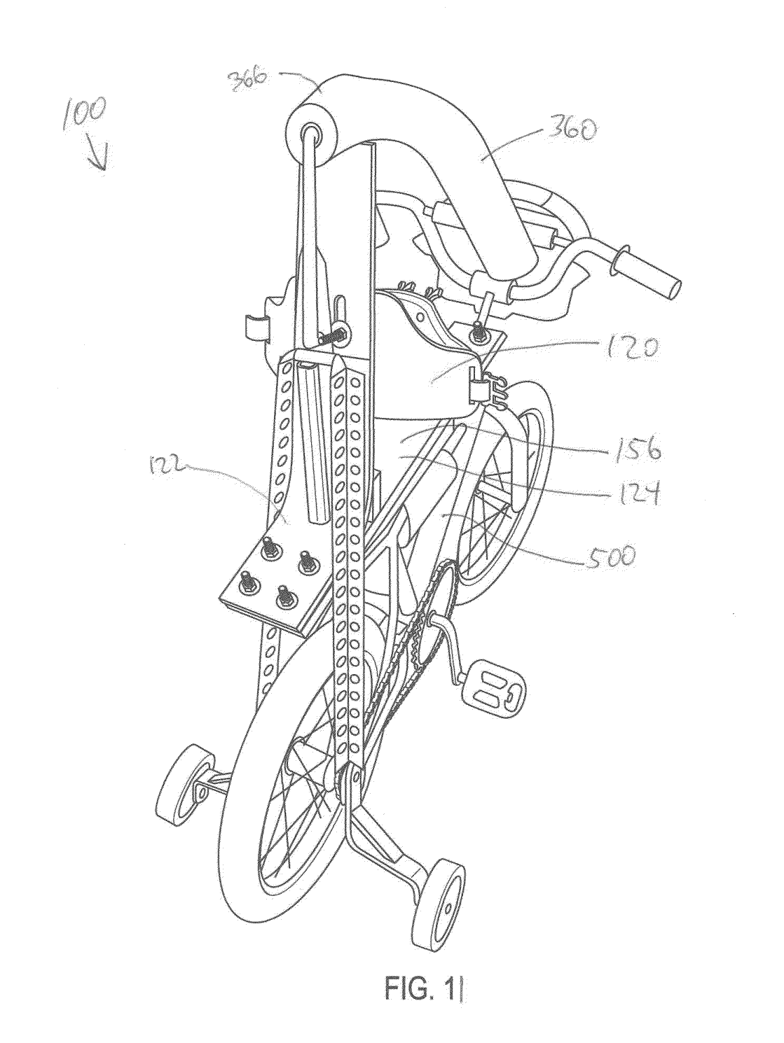

FIG. 11 is a rear perspective view of the orthotic and assistive device of FIG. 8 attached to a bicycle, according to one aspect.

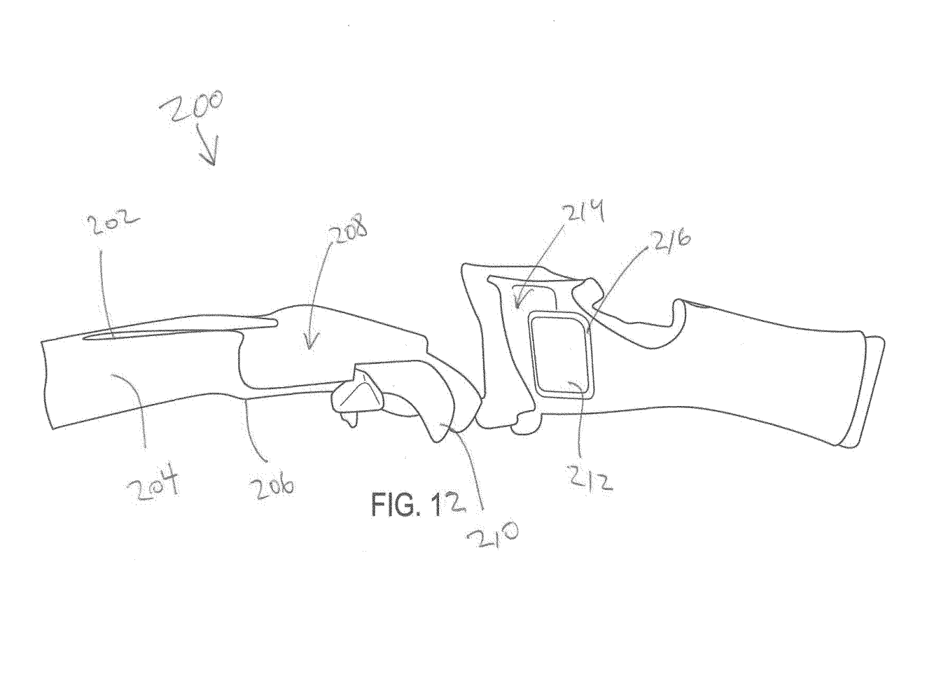

FIG. 12 is a perspective view of a plurality of hand attachment members of the device of FIG. 8, according to one aspect.

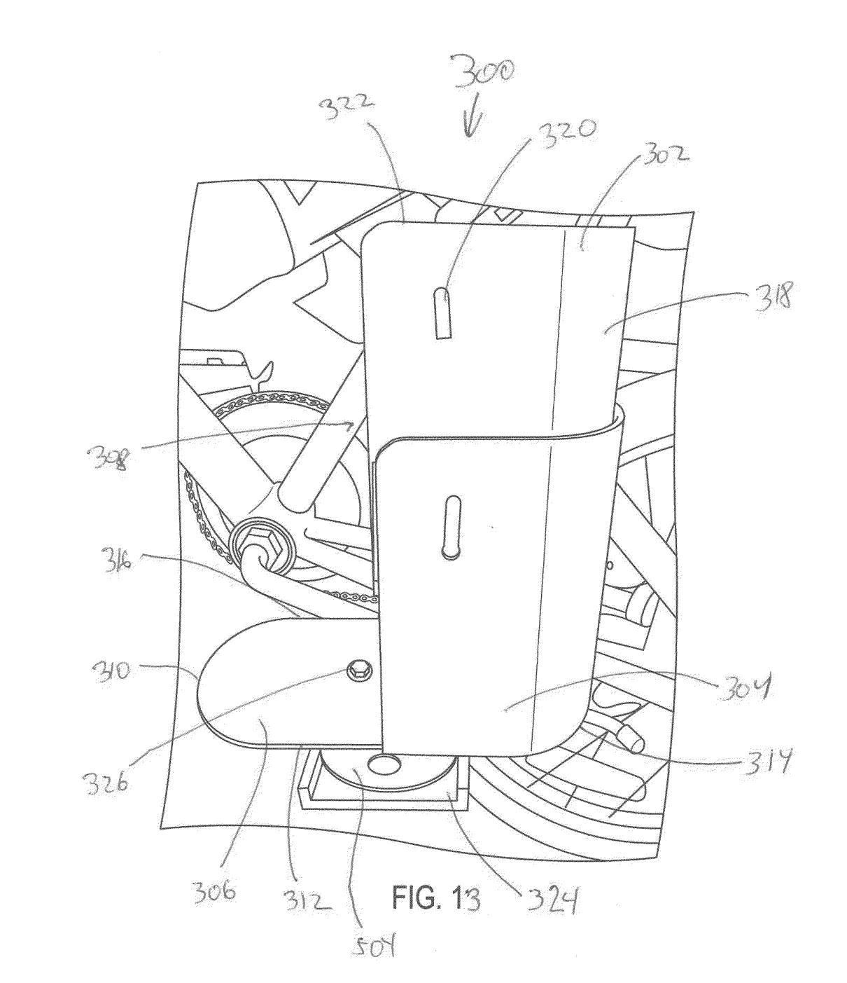

FIG. 13 is a perspective view of the foot attachment member of the device of FIG. 8, according to one aspect.

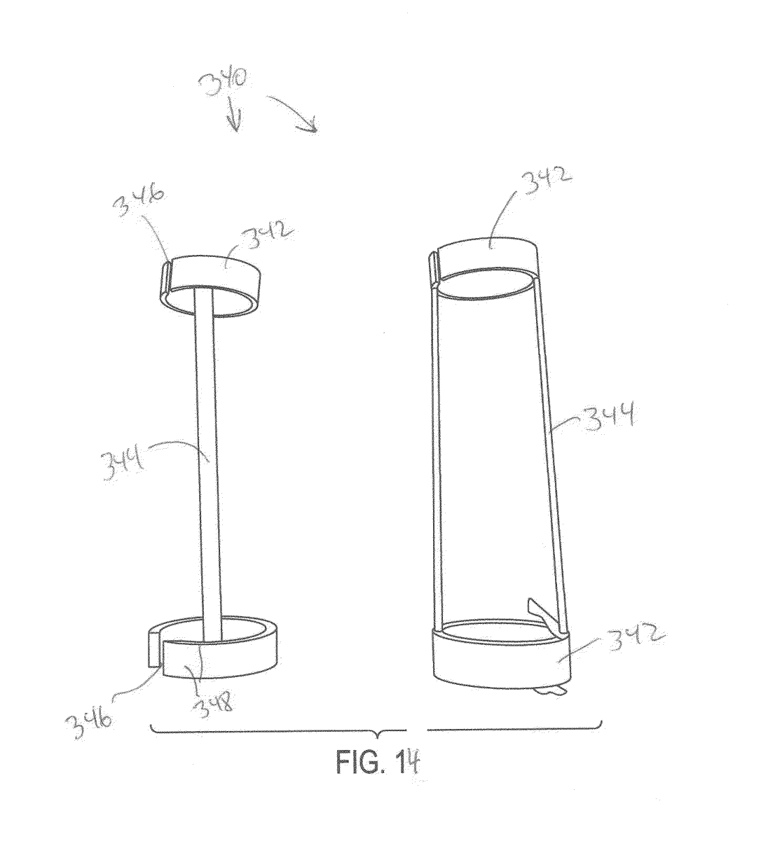

FIG. 14 is a perspective view of a plurality of arm supports of the device of FIG. 8, according to one aspect.

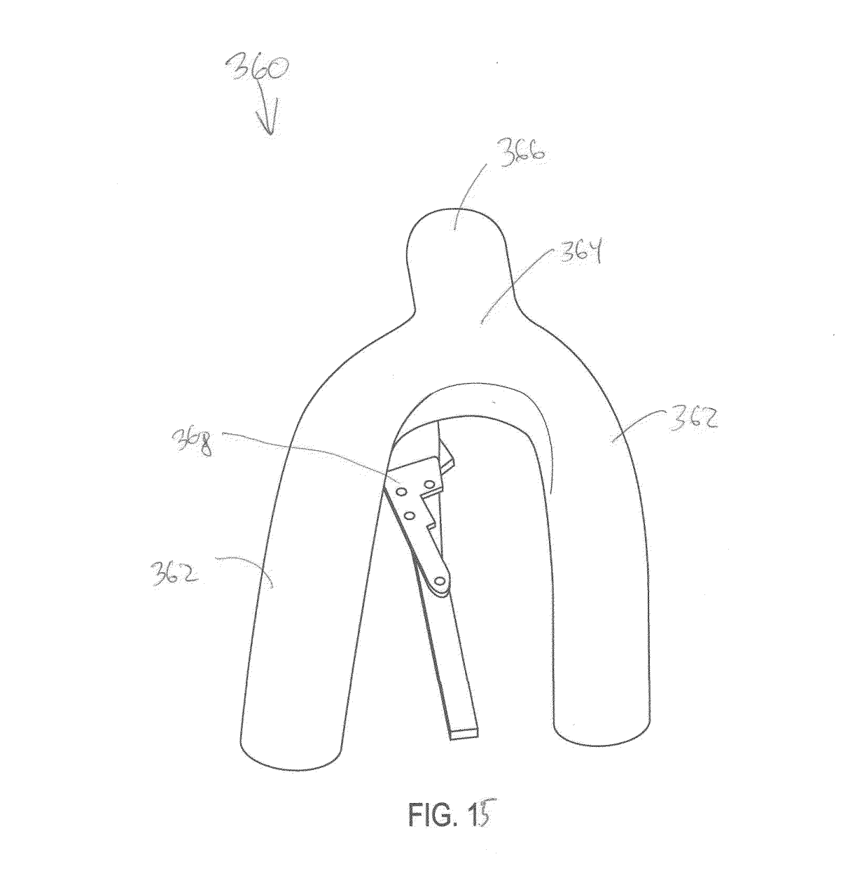

FIG. 15 is a view of a yoke of the device of FIG. 8, according to one aspect.

### DETAILED DESCRIPTION OF VARIOUS EMBODIMENTS

The present systems and apparatuses and methods are understood more readily by reference to the following detailed description, examples, drawing, and claims, and their previous and following descriptions. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description is provided as an enabling teaching in its best, currently known embodiment. To this end, those skilled in the relevant art will recognize and appreciate that many changes can be made to the various aspects described herein, while still obtaining the beneficial results of the technology disclosed. It will also be apparent that some of the desired benefits can be obtained by selecting some of the features while not utilizing others. Accordingly, those with ordinary skill in the art will recognize that many modifications and adaptations are possible, and may even be desirable in certain circumstances, and are a part of the invention described. Thus, the following description is provided as illustrative of the principles of the invention and not in limitation thereof.

As used throughout, the singular forms “a,” “an” and “the” include plural referents unless the context clearly dictates otherwise. Thus, for example, reference to “a component” can include two or more such components unless the context indicates otherwise. Also, the words “proximal” and “distal” are used to describe items or portions of items that are situated closer to and away from, respectively, a user or operator. Thus, for example, the tip or free end of a device may be referred to as the distal end, whereas the generally opposing end or far end may be referred to as the proximal end.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not. As used herein, the term “facilitate” means to make easier or less difficult and the term “impede” means to interfere with, hinder, or delay the progress.

Although the various embodiments are described with reference to use on a horse and/or a bicycle, the assemblies and methods described herein can be used with any of a variety of animals, vehicles and/or surfaces.

An orthotic and assistive device for persons with disabilities is provided, according to various embodiments of the present application. In one aspect, the orthotic and assistive device can be a low-cost orthotic and assistive device formed by, for example and without limitation, 3D printing. In one example, the orthotic and assistive device can be configured for use on an animal such as, for example and without limitation, a horse during hippotherapy. In another example, the orthotic and assistive device can be configured for use on a conventional vehicle such as a bicycle and the like.

In one embodiment, as illustrated in FIGS. 1-6, the orthotic device 10 can comprise at least one of a torso support element 20, at least one strut 22, and at least one animal engagement member 24. In one aspect, the torso support element comprises a pair of opposed torso members 26, 28 that can define an opening 30 for the body of a user and at least one tether 32 or strap configured to couple the opposed torso members to each other. In one aspect, each torso member 26, 28 can be curved so that when coupled together, the torso support element 20 can be substantially oval-shaped when viewed from above. In another aspect, the at least one tether can have a selectively adjustable tether length so that a space between a first torso member 26 and a second torso member 28 can be adjusted. In this manner, the size of the opening 30 formed between the opposed torso members can be selectively adjusted to accommodate users have different sizes and/or to adjust the level of support provided by the device to the user. For example, the size of the opening can be increased to allow increased independence by the user. In another example, the size of the opening 30 can be decreased to allow decreased independence by the user. In another aspect, the torso support element 20 can have a longitudinal axis L_{T} that bisects the opening.

Each torso member 26, 28 of the torso support element 20 can comprise a support wall 34 that selectively limits movement of the torso of the user, such as lateral, anterior and/or posterior flexion of the hip and/or trunk of the user. Optionally, the torso support element can be sized and shaped to lack support for the pelvis of the user. That is, the torso support element 20 can allow the user to respond to the horse's movements and have anterior-posterior movement at the pelvis of the user. In one aspect, the support wall can have an upper edge 36, an inner surface 38 configured to face the user of the device 10, and an opposed outer surface 40 configured to face away from the user. In another aspect, at least a portion of the support wall can be substantially parallel to the longitudinal axis L_{T} of the opening 30. Each torso member can have a distal end 42 having a distal end wall height, a proximal end 44 having a proximal end wall height and a central portion 46 having a central portion wall height. In another aspect, the distal end wall height, the proximal end wall height, and the central portion wall height can be substantially the same. Optionally, in other aspect, at least one of the distal end wall height and the proximal end wall height can be less than the central portion wall height.

In other aspects, each torso member 26, 28 of the pair of opposed torso members of the torso support element 20 can have a tether attachment point 48. In one aspect, the tether attachment point can be an attachment point configured for attaching the tether 32 to the torso support element 20. For example, the tether attachment point can be a slot 50 defined in the support wall 34 at the distal end 42 and the proximal end 44 of the pair of opposed torso members. The tether attachment point can be configured for receiving a portion of the tether therein. In this aspect, a first end of the tether can be threaded through the slot in each torso member 26, 28 to couple the members together.

The at least one animal engagement member 24 can be configured to rest on the back of an animal, such as a horse while limiting lateral or side to side movement of the device 10 relative to the animal. In one aspect, the animal engagement member can comprise an arm 52 sized and shaped to conform to the animal. In another aspect, the arm can have a bottom surface 54 configured to face the animal and an upper surface 56 opposed to the bottom surface. As can be appreciated, the bottom surface can be positioned on the back of the animal and can stabilize the device 10 without being fixedly attached to the horse. The at least one animal engagement member 24 can have a longitudinal axis L_{A} that is substantially normal to the longitudinal axis L_{T} of the torso support element 20. Optionally, the at least one animal engagement member can comprise two or more animal engagement members, as illustrated in FIGS. 1-6.

The at least one strut 22 can couple the torso support element 20 to the at least one animal engagement member 24. Optionally, the at least one strut 22 can comprise two or more struts, as illustrated in the Figures. In one aspect, each strut can couple an animal engagement member to a torso member 26, 28. For example, if two animal engagement members 24 are provided, two struts can also be provided. In one aspect, the at least one strut can have a first end 58 fixedly attached to a portion of the upper surface 56 of the animal engagement member 24 and a second end 60 fixedly attached to a portion of the outer surface 40 of the torso support element. In one aspect, at least one of the strut 22, the torso support element 20, and the animal engagement member can be monolithically formed. Optionally, in another aspect, at least one of the strut, the torso support element, and the animal engagement member 24 can be fixedly attached together with fasteners, such as screws, nuts and bolts, rivets and the like. The first end of the at least one strut can have a first end longitudinal axis L₁ and the second end of the strut can have a second end longitudinal axis L₂. In one aspect, the first end longitudinal axis L₁ can be substantially normal to the second end longitudinal axis L₂. In another aspect, the first end longitudinal axis L₁ can be at an acute angle relative to the second end longitudinal axis L₂. In yet another aspect, the longitudinal axis L_{T} of the torso support element 20 can be substantially parallel to the longitudinal axis L₂ of second end 60 of the strut.

In various aspects, the distance between the upper edge 36 of the support wall 34 of the torso support element 20 and the upper surface 56 of the animal engagement member 24 can be selectively adjustable. For example, in one aspect, the second end 60 of the strut 22 and/or the support wall can define a slot 62 so that the position of the upper edge 36 of the support wall 34 relative to the upper surface 56 of the animal engagement member 24 can be adjusted before being fixedly attached with a fastener 64 extending through the slot. Thus, the fastener can selectively secure the torso members 26, 28 to the strut 22 such that the upper edge of the support wall is a desired distance from the upper surface 56 of the animal engagement member 24. In use, the position of the upper edge 36 can be adjusted by loosening the fastener or other clamping device, adjusting the upper edge of the support wall 34 into the desired position, and tightening the fastener 64 or other clamping device to secure the upper edge in the desired position.

It is contemplated that any of the surfaces of the orthotic device 10 configured to contact the user and/or the animal can be covered with padding 66. For example, at least a portion of the inner surface 38 of the support wall 34 can be covered with padding. In another example, at least a portion of the bottom surface 54 of the animal engagement member 24 can be covered with padding to be more comfortable for the animal. In one aspect, the padding can be a conventional padding such as foam, a blanket and the like.

In one aspect, the orthotic device 10 can further comprise a head support element 68. In this aspect, the head support element can be sized and shaped to support the head of the user. The head support element can comprise a head support wall 70 having an upper edge 72, an inner surface 74 configured to face the user of the device 10, and an opposed outer surface 76 configured to face away from the user. In another aspect, at least a portion of the head support wall can be substantially parallel to the longitudinal axis L_{T} of the opening 30. The head support element 68 can have a distal end 78 having a distal end head wall height, a proximal end 80 having a proximal end head wall height and a central portion 82 having a central portion head wall height. In another aspect, the distal end head wall height, the proximal end head wall height, and the central portion head wall height can be substantially the same. Optionally, in other aspect, at least one of the distal end head wall height and the proximal end head wall height can be less than the central portion head wall height. In further aspects, a tether attachment passage 86 can be defined in the distal end 78 and/or the proximal end 80 of the head support element. The tether attachment passage can be configured so that a tether from for example and without limitation, a helmet of the user can be coupled to the head support element.

The head support element 68 can be spaced from the upper edge 36 of the torso support element 20 a predetermined distance. In one aspect, the head support element comprises a spacer bar 84 configured to position the head support element the predetermined distance. Optionally, the distance from the head support element 68 and the upper edge of the torso support element can be selectively adjustable by a user. For example, in one aspect, the second end 60 of the strut 22 and/or the support wall can define a slot so that the height of the spacer bar 84 extending above the upper edge 36 of the torso support element can be adjusted before being fixedly attached with a fastener 64 extending through the slot. Thus, the fastener can selectively secure the spacer bar to the strut 22 such that the head support element is a desired distance from the torso support element. In use, the position of the head support element 68 can be adjusted by loosening the fastener or other clamping device, adjusting the spacer bar into the desired position, and tightening the fastener 64 or other clamping device to secure the head support element in the desired position. Alternatively, the distance between the head support element 68 and the upper edge 36 of the torso support element 20 can be fixed. In another aspect, the head support can be formed monolithically with least one of the strut 22, the torso support element 20, and the animal engagement member 24. Optionally, in another aspect, at least one of the head support element 68 and the spacer bar 84 can be fixedly attached to the torso member and/or the strut 22 with fasteners, such as screws, nuts and bolts, rivets and the like.

The head support element 68 can rotatable about a head support longitudinal axis L_{H}. That is, the orientation of the head support element can be selectively adjusted by a user relative to the front of the device 10 to provide more or less support to the head of the user as desired. In one aspect, the head support longitudinal axis L_{H} can be substantially parallel to the longitudinal axis L_{T} of the torso support element.

In one aspect, the orthotic device 10 can further comprise a wedge system 88 to allow accommodation of the device 10 to different size animals and to promote appropriate posture for the user on the animal. For example, at least a portion of the wedge system can be positioned between the at least one strut 22 and the animal engagement member 24 to alter the position of the longitudinal axis L_{T} of the torso support element 20 relative to the longitudinal axis L_{A} of the animal engagement member. In another example, at least a portion of the wedge system 88 can be positioned between the at least one strut 22 and a torso member 26, 28 to alter the position of the longitudinal axis L_{T} of the torso support element 20 relative to the longitudinal axis L_{A} of the animal engagement member 24. The wedge system can allow accommodation of the orthotic device 10 to different size animals and to promote appropriate posture for the user on the animal.

In one aspect, any and/or all of the components of the orthotic device 10 can be formed relatively quickly and/or relatively inexpensively when compared to conventional orthotic “saddles.” For example, at least one of the torso support element 20, the strut 22, and the animal engagement member 24 can be formed with 3D (or “additive”) printing. In one aspect, the orthotic device 10 can be designed using a computer aided design program and printed with a flexible plastic on a 3D printer. With 3D printing, users can create an assistive orthotic device 10 to promote standardization of side walker support, increase autonomy of the patient and improving safety during each session. 3D printing promotes construction of multiple devices in fast, efficient and economical ways. Recent improvements in 3D technology have decreased the cost of the printers and manufacturing, expanding access across the medical community. Using this technique, the orthotic device of the present application can be produced for about $150, whereas conventional saddles for use in hippotherapy can cost around $5,000. Optionally, at least one of the torso support element 20, the strut 22, and the animal engagement member 24 can be formed from polymeric materials such as nylon, carbon fiber and the like and/or metallic materials such as stainless steel and the like.

An orthotic device 10 according to this embodiment can be assembled to comprise any or all of the components as described above. In one aspect, each torso member 26, 28 of the pair of opposed torso members of the torso support element 20 can be placed around the torso of a user so that the torso of the user extends through the opening 30 of the torso support element. The at least one tether 32 can be adjusted so that the opening has a predetermined size and thus, provides a desired level of support to the user. Optionally, the distance between the upper edge 36 of the support wall and the upper surface 56 of the animal engagement member 24 can be adjusted to a predetermined height by sliding the strut 22 relative to the torso support element and fixedly attaching the strut in the desired position with the fastener 64. The user and the device 10 can be placed on the animal, such as for example and without limitation, a horse, so that the bottom surface of the animal engagement member 24 contacts the back of the animal. The user can then be led around by a side walker during a hippotherapy session.

To test the effectiveness of the orthotic device 10, two users each completed one 100 foot lap mounted on a horse without the assistive device and with side walker support as needed to maintain an erect sitting posture and appropriate head control. The user then donned the orthotic device and completed another lap on the same horse. Dependence level and side walker assistance was collected utilizing a functional level scale. The outcome measures used in this study was the Functional Level Scale (FLS) and hands needed for support. The outcome measures determined the functional level of each participant and assistance given by side walkers during a hippotherapy session with and without the use of the device.

As can be appreciated, use of the orthotic device 10 can decrease the user's reliance on a side walker, thereby increasing the safety of the user and the side walker. Further, the user can demonstrate improvement in trunk and head control and amount of assistance required by side walkers with the device 10 in use as compared to without the device. Users can became more actively engaged in the session with decreased side walker interference. Also users can require less assistance given by the side walkers.

This study examined the functional effects of a low cost assistive device designed and created using a 3D printer. The orthotic device 10 can be adjustable in diameter and height to accommodate users of various sizes, ages and disabilities. Optionally, portions of the orthotic device such as, for example and without limitation, the torso support element 20 can be provided in different sizes to accommodate users of various sizes, ages and disabilities. In one aspect, the torso support element can be interchanged (for example, to a smaller or larger torso support element 20) to allow the user to control more or less of their own movement or to promote more or less stability. Thus, the support offered by the device can be increased or decreased according the user's core stability and balance responses, providing just enough support but not too much support to promote motor learning. Side walker assistance was directed toward safety and prevented “too much assistance.” The ability to decrease the input provided by side walkers allowed the user improved opportunity to problem-solve. The functional improvements during the hippotherapy session could promote further motor learning. Development of the device promoted better student understanding of posture and biomechanics through the design process.

Results from the study suggest that physical therapists can use 3D printing to create a low cost adjustable assistive device to decrease side walker exertion and improve user positioning during hippotherapy. The device has proven to be effective in improving the subject's functional level during the hippotherapy session. The orthotic device 10 can also help with the standardization of side walker support, which allowed the subjects to be more actively involved in motor play, promoting growth and development and supporting physical therapy interventions.

In a second embodiment, as illustrated in FIG. 7, the orthotic device 10 can comprise the at least one of a torso support element 20 and the at least one strut 22 as previously described. Instead of an animal engagement member 24, however, in one aspect, the orthotic device can further comprise at least one planar engagement member 90. In this aspect, the planar member can be configured to rest on a flat surface, such as a floor, table and the like. In another aspect, the planar engagement member can comprise a substantially flat planar arm 92. That is, the flat planar arm can have a substantially flat bottom surface 94 configured to face the planar surface and an upper surface 96 opposed to the bottom surface. As can be appreciated, the bottom surface can be positioned on the flat surface and can stabilize the device 10 without being fixedly attached to the flat surface. The at least one planar engagement member 90 can have a longitudinal axis L_{P} that is substantially normal to the longitudinal axis L_{T} of the torso support element 20. Optionally, the at least one planar engagement member can comprise two or more planar engagement members, as illustrated in FIG. 7.

The orthotic device 10 of FIG. 7 can be assembled as previously described for the device of FIGS. 1-6. In use, both the first torso member 26 and the second torso member 28 of the orthotic device of FIG. 7 can be provided for more involved users. Optionally, just the first torso member or the second torso member can be provided depending on the needs of the user. The orthotic device of FIG. 7 is light, flexible and allows users to engage in seated activities with hands free for play. This device 10 can also be attached to chairs to allow a user to engage in mealtimes at home or a restaurant and work at a desk at school. The orthotic device of FIG. 7 also can allow a therapist to engage the child in upper extremity play freeing the therapist to focus on hand development.

In a third embodiment, as illustrated in FIG. 8-15, the orthotic device 100 can comprise at least one of a torso support element 120, at least one strut 122, at least one bicycle engagement member 124, a hand attachment member 200, and a foot attachment member 300 configured to assist the user in riding a conventional bicycle 500.

In one aspect, the torso support element 120 comprises a pair of opposed torso members 126, 128 that can define an opening 130 for the body of a user and at least one tether 132 or strap configured to couple the opposed torso members to each other. In one aspect, each torso member 126, 128 can be curved so that when coupled together, the torso support element 120 can be substantially oval-shaped when viewed from above. In another aspect, the at least one tether can have a selectively adjustable tether length so that a space between a first torso member 126 and a second torso member 128 can be adjusted. In this manner, the size of the opening 130 formed between the opposed torso members can be selectively adjusted to accommodate users have different sizes and/or to adjust the level of support provided by the device to the user. For example, the size of the opening can be increased to allow increased independence by the user. In another example, the size of the opening 130 can be decreased to allow decreased independence by the user. In another aspect, the torso support element 120 can have a longitudinal axis L_{T} that bisects the opening.

Each torso member 126, 128 of the torso support element 120 can comprise a support wall 134 that selectively limits movement of the torso of the user, such as lateral, anterior and/or posterior flexion of the hip and/or trunk of the user. Optionally, the torso support element can be sized and shaped to lack support for the pelvis of the user. That is, the torso support element 120 can allow the user to respond to the horse's movements and have anterior-posterior movement at the pelvis of the user. In one aspect, the support wall can have an upper edge 136, an inner surface 138 configured to face the user of the device 100, and an opposed outer surface 140 configured to face away from the user. In another aspect, at least a portion of the support wall can be substantially parallel to the longitudinal axis L_{T} of the opening 130. Each torso member can have a distal end 142 having a distal end wall height, a proximal end 144 having a proximal end wall height and a central portion 146 having a central portion wall height. In another aspect, the distal end wall height, the proximal end wall height, and the central portion wall height can be substantially the same. Optionally, in other aspect, at least one of the distal end wall height and the proximal end wall height can be less than the central portion wall height.

In other aspects, each torso member 126, 128 of the pair of opposed torso members of the torso support element 120 can have a tether attachment point 148. In one aspect, the tether attachment point can be an attachment point configured for attaching the tether 132 to the torso support element 120. For example, the tether attachment point can be a slot 150 defined in the support wall 134 at the distal end 142 and the proximal end 144 of the pair of opposed torso members. The tether attachment point can be configured for receiving a portion of the tether therein. In this aspect, a first end of the tether can be threaded through the slot in each torso member 126, 128 to couple the members together.

The at least one bicycle engagement member 124 can be configured to securely couple the device 100 to a bicycle 500. In one aspect, the bicycle engagement member can comprise an elongate rod 152 having a proximal end sized and shaped to be inserted into the original seat tube of the bicycle. In another aspect, the bicycle engagement member 124 can further comprise a seat attachment member 154 configured to be coupled to a distal end 155 of the elongate rod 152. In one aspect, the bicycle engagement member can further comprise a base 156 coupled to the seat attachment member. For example, the seat attachment member can be positioned on the distal end of the elongate rod, and the base can be positioned on the seat attachment member. In this aspect, an upper surface 157 of the base can be positioned in a plane substantially normal to a longitudinal axis L_{R} of the elongate rod 152. Optionally, the upper surface of the base 156 can be positioned in a plane that is at an acute angle relative to the longitudinal axis L_{R} of the elongate rod. In one aspect, the angle formed between the upper surface 157 of the base and the longitudinal axis L_{R} of the elongate rod 152 can be selectively adjustable by the user.

The at least one strut 122 can couple the torso support element 120 to the at least one bicycle engagement member 124. Optionally, the at least one strut 122 can comprise two or more struts, as illustrated in the Figures. In one aspect, each strut can couple a portion of the bicycle engagement member to a torso member 126, 128. For example, a first strut can be positioned on a distal end of the upper surface 157 of the base 156 or the seat attachment member 154 of the bicycle engagement member, and a second strut can be positioned on a proximal end of the upper surface of the base. In one aspect, the at least one strut can have a first end 158 fixedly attached to a portion of the upper surface 157 of the bicycle engagement member 124 or the seat attachment member 154 and a second end 160 fixedly attached to a portion of the outer surface 140 of the torso support element. In one aspect, at least one of the strut 122, the torso support element 120, and the bicycle engagement member can be monolithically formed. Optionally, in another aspect, at least one of the strut, the torso support element, and the bicycle engagement member 124 can be fixedly attached together with fasteners, such as screws, nuts and bolts, rivets and the like. The first end of the at least one strut can have a first end longitudinal axis L₁ and the second end of the strut can have a second end longitudinal axis L₂. In one aspect, the first end longitudinal axis L₁ can be substantially normal to the second end longitudinal axis L₂. In another aspect, the first end longitudinal axis L₁ can be at an acute angle relative to the second end longitudinal axis L₂. In yet another aspect, the longitudinal axis L_{T} of the torso support element 120 can be substantially parallel to the longitudinal axis L₂ of second end 160 of the strut.

In various aspects, the distance between the upper edge 136 of the support wall 134 of the torso support element 120 and the upper surface 157 of the bicycle engagement member 124 can be selectively adjustable. For example, in one aspect, the second end 160 of the strut 122 and/or the support wall can define a slot 162 so that the position of the upper edge 136 of the support wall 134 relative to the upper surface 157 of the bicycle engagement member 124 can be adjusted before being fixedly attached with a fastener 164 extending through the slot. Thus, the fastener can selectively secure the torso members 126, 128 to the strut 122 such that the upper edge of the support wall is a desired distance from the upper surface 157 of the bicycle engagement member 124. In use, the position of the upper edge 136 can be adjusted by loosening the fastener or other clamping device, adjusting the upper edge of the support wall 134 into the desired position, and tightening the fastener 164 or other clamping device to secure the upper edge in the desired position.

It is contemplated that any of the surfaces of the orthotic device 100 configured to contact the user and/or the bicycle can be covered with padding 166. For example, at least a portion of the inner surface 138 of the support wall 134 can be covered with padding. In one aspect, the padding can be a conventional padding such as foam, a blanket and the like.

In one aspect, the orthotic device 100 can further comprise a head support element 168. In this aspect, the head support element can be sized and shaped to support the head of the user. The head support element can comprise a head support wall 170 having an upper edge 172, an inner surface 174 configured to face the user of the device 100, and an opposed outer surface 176 configured to face away from the user. In another aspect, at least a portion of the head support wall can be substantially parallel to the longitudinal axis L_{T} of the opening 130. The head support element 168 can have a distal end 178 having a distal end head wall height, a proximal end 180 having a proximal end head wall height and a central portion 182 having a central portion head wall height. In another aspect, the distal end head wall height, the proximal end head wall height, and the central portion head wall height can be substantially the same. Optionally, in other aspect, at least one of the distal end head wall height and the proximal end head wall height can be less than the central portion head wall height. In further aspects, a tether attachment passage 186 can be defined in the distal end 178 and/or the proximal end 180 of the head support element. The tether attachment passage can be configured so that a tether from for example and without limitation, a helmet of the user can be coupled to the head support element.

The head support element 168 can be spaced from the upper edge 136 of the torso support element 120 a predetermined distance. In one aspect, the head support element comprises a spacer bar 184 configured to position the head support element the predetermined distance. Optionally, the distance from the head support element 168 and the upper edge of the torso support element can be selectively adjustable by a user. For example, in one aspect, the second end 160 of the strut 122, the spacer bar 184 and/or the support wall can define a slot so that the height of the spacer bar 184 extending above the upper edge 136 of the torso support element can be adjusted before being fixedly attached with a fastener 164 extending through the slot. Thus, the fastener can selectively secure the spacer bar to the strut 122 such that the head support element is a desired distance from the torso support element. In use, the position of the head support element 168 can be adjusted by loosening the fastener or other clamping device, adjusting the spacer bar into the desired position, and tightening the fastener 164 or other clamping device to secure the head support element in the desired position. Alternatively, the distance between the head support element 168 and the upper edge 136 of the torso support element 120 can be fixed. In another aspect, the head support can be formed monolithically with least one of the strut 122, the torso support element 120, and the bicycle engagement member 124. Optionally, in another aspect, at least one of the head support element 168 and the spacer bar 184 can be fixedly attached to the torso member and/or the strut 122 with fasteners, such as screws, nuts and bolts, rivets and the like.

The head support element 168 can rotatable about a head support longitudinal axis L_{H}. That is, the orientation of the head support element can be selectively adjusted by a user relative to the front of the device 100 to provide more or less support to the head of the user as desired. In one aspect, the head support longitudinal axis L_{H} can be substantially parallel to the longitudinal axis L_{T} of the torso support element.

In one aspect, the orthotic device 100 can further comprise a wedge system 188 to allow accommodation of the device to different size bicycles and to promote appropriate posture for the user on the bicycle. For example, at least a portion of the wedge system can be positioned between the at least one strut 122 and the bicycle engagement member 124 to alter the position of the longitudinal axis L_{T} of the torso support element 20 relative to the longitudinal axis L_{R} of the elongate rod of bicycle engagement member. In another example, at least a portion of the wedge system 188 can be positioned between the at least one strut 122 and a torso member 126, 128 to alter the position of the longitudinal axis L_{T} of the torso support element 120 relative to the longitudinal axis L_{R} of the elongate rod of the bicycle engagement member 124.

The hand attachment member 200 can comprise an at least partially open shell 202 sized, shaped and/or contoured to contain a portion of a hand and/or arm of the user therein. For example, the shell can have at least one sidewall 204 extending away from a bottom wall 206. The at least one sidewall and the bottom wall can define a channel 208 in the hand attachment member that can be configured to contain a portion of a hand and/or arm of the user. In one aspect, a distal end 210 of the hand attachment member can be curved as the hand of a bicycle rider would be when grasping the handlebar 502 of the bicycle. That is, the hand attachment member 200 can be sized and shaped so that when a portion of the hand of the user is positioned in the channel, the hand attachment member places the hand in a desired position relative to the bicycle 500. In another aspect, the hand attachment member 200 can further comprise a handlebar attachment element 212. For example, the handlebar attachment element can be a groove 214 defined in the bottom wall 206 of the hand attachment member. Optionally, in another aspect, a shoulder 216 can extend away from the bottom wall in a direction opposed to the at least one sidewall, and the groove can be defined therein the shoulder. In a further aspect, the groove 214 can be sized and shaped to contain a portion of the handlebar 502 of the bicycle therein. In use, described more fully below, a portion of the handlebar can snap-fit into the groove 214 to secure the hand attachment member 200 to the bicycle. Optionally, the hand attachment member can be coupled to the handlebar with a tether, strap and the like. Similarly, a portion of the hand and/or arm of the user can be coupled to the hand attachment member with a tether, strap and the like so that the hand of the user remains positioned in the channel 208 of the hand attachment member.

In one aspect, the hand attachment member 200 can be configured to keep the hand and/or wrist of the user in a functional grasp position as a conventional bicycle rider would use. Coupling of the hand attachment member to the bicycle 500 and the user can allow the user to steer the bicycle. Further, the shape of the hand attachment member 200 can be selected to alter the position of the user relative to the bicycle into a more desired position.

The foot attachment member 300 is configured to couple the user of the bicycle 500 to a pedal 504 of the bicycle and to maintain the feet, knee, and hip of the user in conventional alignment. In one aspect, the foot attachment member can comprise an at least partially open sleeve 302 sized and shaped to contain a portion of a foot and/or leg of the user therein. For example, the sleeve can have at least one sidewall 304 extending away from a bottom wall 306 a predetermined distance. The at least one sidewall and the bottom wall can define a channel 308 in the foot attachment member that can be configured to contain a portion of a leg and/or foot of the user. In another aspect, the at least one sidewall 304 of the foot attachment member 300 can comprise a first sidewall that extends from a perimeter edge 310 of a portion of the bottom wall. For example, the first sidewall can extend from at least a portion of a first side 312 of the bottom wall, a second side 314 of the bottom wall, and a third side 316 of the bottom wall.

In a further aspect, the at least one sidewall 304 can comprise a second sidewall 318 configured to nest therein the channel 308 and that can slide relative to the first sidewall. That is, the second sidewall can slide longitudinally relative to the first sidewall to change the effective height of the sidewall. For example, the second sidewall 318 can slide toward the bottom wall 306 to shorten the height of the foot attachment member 300, and the second sidewall can slide away from the bottom wall to increase the height of the foot attachment member. At least one bore or slot 320 can be defined in a portion of the first sidewall 304 and the second sidewall 318 so that the position of the second sidewall relative to the first sidewall can be adjusted before being fixedly attached with a fastener or strap extending through the slot 320. Thus, the fastener can selectively secure the second sidewall 318 to the first sidewall 304 such that an upper edge 322 of the second sidewall is a desired distance from the bottom wall 306 of the foot attachment member 300. In use, at least a portion of the foot and leg of the user can be positioned therein the channel 308 and the position of the upper edge of the second sidewall 318 can be adjusted to a desired position relative to the first sidewall 304. In the desired position, the fastener or strap can be tightened to secure the upper edge 322 in the desired position and to secure the leg of the user to the foot attachment member 300. In use, the position of the upper edge 322 of the second sidewall can be selected to prevent or reduce adduction at the hip of the user while pedaling to help maintain a more neutral hip position.

In one aspect, the foot attachment member 300 can further comprise a pedal plate 324 configured to fixedly attach the foot attachment member to the pedal 504 of the bicycle 500. For example, the pedal plate and the bottom wall 306 of the foot attachment member can each define at least one bore 326. When the at least one bore of the bottom wall is aligned with the at least one bore of the pedal plate, a fastener, such as a screw, nut and bolt and the like can be inserted through the bore to fixedly attach the pedal plate 324 to the bottom wall. In use, the bottom wall 306 can be positioned above the pedal of the bicycle, and the pedal plate can be positioned below the pedal 504 such that the bores 326 are aligned. The fastener can be inserted through the aligned bores and the pedal to fixedly attach the foot attachment member 300 to the pedal.

In one aspect, the orthotic device 100 of this embodiment can further comprise at least one arm support 340 and a yoke 360. The at least one arm support be configured to keep the arm of the user extended so that the wrist and/or hand stay in a function grasp position and to keep a user with flexor tone from pulling into flexor pattern. In another aspect, the at least one arm support 340 can comprise a plurality of collars 342 spaced by at least one rod 344. For example, in use, the user can insert his hand through each collar of the plurality of collars so that a first collar is near the wrist of the user and a second collar is near the upper arm of the user. The at least one rod can be a rigid rod that prevents or restricts rotation of the elbow of the user. In one aspect, each collar 342 can define a slit 346 extending through the collar to allow the collar to flex radially so that the hand of the user can be more easily inserted through the collar. In another aspect, at least a portion of at least one collar 342 can be covered with a hook and loop fastener 348 so that the arm support 340 can be fastened to the bicycle and/or other components of the orthotic device 100.

The yoke 360 is configured to provide support to hold the upper trunk of the user in an upright position and to provide head support to the user. In one aspect, the yoke is a “Y” or “U” shaped element having two arms 362 extending a central hub 364. The arms can extend away from the hubs at an angle so that a portion of the head and/or neck of the user can be positioned between the arms. A connecting leg 366 can extend from the central hub in a direction generally away from the arms. The connecting leg can be configured to attach the yoke 360 to the bicycle 500. In one aspect, the connecting leg can have a leg length so that the yoke is at a desired height relative to the user. In another aspect, the leg length of the connecting leg 366 can be adjustable to allow the yoke to be positioned at different heights as desired. In a further aspect, the connecting leg comprise at least one hinge 368 or other flexible member. The hinge can be a strap hinge, a barrel hinge, a pivot hinge and the like. In this aspect, the connecting leg can be hingedly attached to the central hub 364 and/or to the bicycle so that the yoke can be rotated into position on the user.

It is contemplated that any of the surfaces of the orthotic device 100 of this embodiment configured to contact the user can be covered with padding 166 to be more comfortable for the user. For example, at least a portion of the inner surface 138 of the torso support element 120, the hand attachment member 200 and/or the yoke 360 can be covered with padding such as for example and without limitation, foam.

In one aspect, any and/or all of the components of the orthotic device 100 can be formed relatively quickly and/or relatively inexpensively when compared to conventional orthotic attachment device. For example, at least one of the hand attachment member 200 and the foot attachment member 300 can be formed with 3D (or “additive”) printing. In one aspect, the orthotic device 100 can be designed using a computer aided design program and printed with a flexible plastic on a 3D printer. In another aspect, at least a portion of the elements of the device can be formed at one time on the 3D printer and separated later for assembly. In still another aspect, the relatively fast speed and inexpensive costs of 3D printing can allow components to be custom made for each user. For example, fit measurements can be taken of a user and the components can be custom-made to fit that user precisely. Recent improvements in 3D technology have decreased the cost of the printers and manufacturing, expanding access across the medical community. Using this technique, the orthotic device of the present application can be produced for about $150, whereas conventional vehicle modifications can cost much more. Optionally, at least one of the torso support element 120, the hand attachment member 200 and the foot attachment member 300 can be formed from polymeric materials such as nylon, carbon fiber and the like and/or metallic materials such as stainless steel and the like.

An orthotic device 100 according to this embodiment can be assembled to comprise any or all of the components as described above. In one aspect, the torso support element 120 can be coupled to the seat tube of the bicycle 500 by the bicycle engagement member 124. As known to one of skill in the art, rods, beams and the like can be added to the bicycle as necessary to support components of the orthotic device. The foot attachment member 300 can be coupled to the pedal 504 and the hand attachment member 200 can be coupled to the handlebar 502, as described above. A user can sit on the seat of the bicycle and each torso member 126, 128 of the pair of opposed torso members of the torso support element 120 can be placed around the torso of the user so that the torso of the user extends through the opening 130 of the torso support element. The size of the opening can be adjusted with the at least one tether 132 or, optionally, positioning the torso members in an overlying position relative to each other and fastening them in place. A portion of the foot and/or leg of the user can be positioned in the sleeve 302 of the foot attachment member 300. The distance between the upper edge 322 of the second sidewall 318 of the foot attachment member can be adjusted to a predetermined height by sliding the second sidewall relative to the first sidewall 304 and fixedly attaching the second sidewall and the foot in the desired position with a strap and the like. A portion of the hand and/or arm of the user can be inserted through the collars 342 of the arm support 340. A portion of the hand and/or arm can then be positioned in the channel 208 of the hand attachment member 200 and fixedly attached with a strap and the like. The yoke 360 can be rotated into position around the head and/or neck of the user. The user can then pedal the bicycle 500 conventionally.

It is contemplated that certain users may not require the use of all of the components described herein, and that the device 100 can function with only one, a plurality or all of the components as described above. For example, certain users might not require the use of the torso support element, 120, the hand attachment member 200, the foot attachment member 300, the arm support 340 and/or the yoke 360. In this case, the device can be assembled with only the components necessary for that particular user.

To test the effectiveness of the orthotic device 100, five users were positioned on a bicycle 500 with and without the orthotic device. Each user could ride the bicycle farther, faster and for a longer time with the orthotic device 100 on compared to without using the device. Users of the device also required fewer people assisting.

As can be appreciated, with advances in 3D printing, a user can be provided an opportunity to participate in normalized play in a time efficient, cost effective and productive manner. As a result, the user may gain the advantages of increased independence, range of motion, strength, neuromuscular development, and social and psychological development.

Although the assemblies and methods are described herein in the context of an orthotic device for use in hippotherapy, cycle therapy, and surface therapy, the technology disclosed herein is also useful and applicable in other contexts. Moreover, although several embodiments have been described herein, those of ordinary skill in art, with the benefit of the teachings of this disclosure, will understand and comprehend many other embodiments and modifications for this technology. The invention therefore is not limited to the specific embodiments disclosed or discussed herein, and that can other embodiments and modifications are intended to be included within the scope of the appended claims. Moreover, although specific terms are occasionally used herein, as well as in the claims or concepts that follow, such terms are used in a generic and descriptive sense only, and should not be construed as limiting the described invention or the claims that follow.

What is claimed is:

1. An orthotic device for use in hippotherapy comprising:
a torso support element comprising a pair of opposed torso members that are spaced from each other to define an opening for a torso of a user and at least one tether configured to couple the opposed torso members to each other, wherein each torso member comprises a support wall configured to selectively limit movement of the torso of the user and wherein the support wall has an upper edge, an inner surface configured to face the user, and an opposed outer surface configured to face away from the user;
at least one animal engagement member comprising an arm sized and shaped to conform to a portion of the back of an animal, wherein the arm has a bottom surface configured to rest on the animal and an opposed upper surface facing away from the animal; and
at least one strut configured to couple the torso support element to the at least one animal engagement member, wherein the at least one strut has a first end fixedly attached to a portion of the upper surface of the animal engagement member and a second end fixedly attached to a portion of the outer surface of the torso support element.

2. The orthotic device of claim 1, wherein each torso member is curved such that when coupled together, the torso support element is substantially oval-shaped when viewed from above.

3. The orthotic device of claim 1, wherein the size of the opening defined between the opposed torso members is selectively adjustable to adjust the level of support provided by the device to the user.

4. The orthotic device of claim 1, wherein the torso support element is configured to allow the user to respond to movements of the animal and have anterior-posterior movement at the pelvis of the user.

5. The orthotic device of claim 1, wherein the bottom surface of the arm stabilizes the device without being fixedly attached to the animal.

6. The orthotic device of claim 1, wherein the at least one animal engagement member comprises a pair of animal engagement members and wherein the at least one strut comprises a pair of struts.

7. The orthotic device of claim 6, wherein a first strut of the pair of struts couples a first animal engagement member of the pair of animal engagement members to a first torso member of the pair of opposed torso members, and wherein a second strut of the pair of struts couples a second animal engagement member of the pair of animal engagement members to a second torso member of the pair of opposed torso members.

8. The orthotic device of claim 1, wherein a distance between the upper edge of the support wall of the torso support element and the upper surface of the at least one animal engagement member is selectively adjustable.

9. The orthotic device of claim 1, further comprising a head support element spaced from the torso support element a predetermined distance by a spacer bar, wherein the head support element is configured to support the head of the user, wherein the head support element comprises a head support wall.

10. The orthotic device of claim 9, wherein a tether attachment passage is defined in a portion of the head support element, and wherein the tether attachment passage is configured so that a tether from a helmet of the user can be coupled to the head support element.

11. The orthotic device of claim 9, wherein a distance between the head support element and the upper edge of the torso support element is selectively adjustable.

12. The orthotic device of claim 9, further comprising a wedge system positioned between the at least one strut and the at least one animal engagement member to alter the position of a longitudinal axis of the torso support element relative to the longitudinal axis of the animal engagement member.

13. The orthotic device of claim 9, further comprising a wedge system positioned between the at least one strut and a torso member of the pair of torso members to alter the position of the longitudinal axis of the torso support element relative to the longitudinal axis of the animal engagement member.

14. The orthotic device of claim 9, wherein the head support element is rotatable about a head support longitudinal axis.

15. The orthotic device of claim 1, wherein each torso member can have a distal end having a distal end wall height, a proximal end having a proximal end wall height and a central portion having a central portion wall height and wherein at least one of the distal end wall height and the proximal end wall height is less than the central portion wall height.

16. A method of hippotherapy comprising:
providing an orthotic device comprising:
a torso support element comprising a pair of opposed torso members that are spaced from each other to define an opening for a torso of a user and at least one tether configured to couple the opposed torso members to each other, wherein each torso member comprises a support wall configured to selectively limit movement of the torso of the user and wherein the support wall has an upper edge, an inner surface configured to face the user, and an opposed outer surface configured to face away from the user;
at least one animal engagement member comprising an arm sized and shaped to conform to a portion of the back of an animal, wherein the arm has a bottom surface configured to rest on the animal and an opposed upper surface facing away from the animal; and
at least one strut configured to couple the torso support element to the at least one animal engagement member, wherein the at least one strut has a first end fixedly attached to a portion of the upper surface of the animal engagement member and a second end fixedly attached to a portion of the outer surface of the torso support element;
positioning the orthotic device on the back of an animal;
positioning the torso of a user in the opening defined by the pair of opposed torso members; and
leading the user and animal around by a side walker.

17. The method of claim 16, wherein a level of support offered by the orthotic device to the user is selectively adjustable.

18. The method of claim 17, wherein the step of providing an orthotic device comprises providing elements of the device in different sizes to accommodate users of various sizes, ages and disabilities.

19. The method of claim 18, wherein the size of the torso support element is interchangeable to allow the user to control more of their own movement.

20. An orthotic device for use in bicycle therapy comprising:
a torso support element comprising a pair of opposed torso members that are spaced from each other to define an opening for a torso of a user and at least one tether configured to couple the opposed torso members to each other, wherein each torso member comprises a support wall configured to selectively limit movement of the torso of the user and wherein the support wall has an upper edge, an inner surface configured to face the user, and an opposed outer surface configured to face from the user;
a bicycle engagement member comprising an elongate rod having a proximal end configured to be inserted into a seat tube of the bicycle and a seat attachment member coupled to a distal end of the elongate rod;
at least one strut configured to couple the torso support element to the bicycle engagement member, wherein the at least one strut has a first end fixedly attached to a portion of the upper surface of the bicycle engagement member and a second end fixedly attached to a portion of the outer surface of the torso support element.
